(19) United States
(12) Patent Application Publication
(10) Pub. No.: US 2023/0268055 A1
(43) Pub. Date: Aug. 24, 2023

(54) SYSTEM AND METHOD FOR BIOMETRIC AND PSYCHOMETRIC BASED CONTENT DISPLAY

(71) Applicant: Sorce Technologies, Inc., Santa Monica, CA (US)

(72) Inventors: Jessica Corbin, Santa Monica, CA (US); Kai Mildenberger, San Francisco, CA (US); Galen Buckwalter, San Francisco, CA (US); Eric Burgess, San Dimas, CA (US); Eugene Russiyanov, San Francisco, CA (US)

(73) Assignee: Sorce Technologies, Inc., Santa Monica, CA (US)

(21) Appl. No.: 18/174,556

(22) Filed: Feb. 24, 2023

Related U.S. Application Data
(60) Provisional application No. 63/268,465, filed on Feb. 24, 2022.

(51) Int. Cl. G16H 20/70; G16H 10/20

(52) U.S. Cl. CPC G16H 20/70; G16H 10/20

(57) ABSTRACT

A method and system for biometric and psychometric based content display. The method includes storing a psychometric assessment instrument as a data structure on a mobile device. The psychometric assessment instrument includes user data for ascertaining a user’s psychometric identity. A server collects and tags content by attribute for each specific classification of the plurality of psychometric classifications. The content is further organized across three state classes and cross-referenced with a plurality of interests. A pulse rate is measured using a camera on the mobile device. The pulse rate is measured using temporal color contrast between two frames. The pulse rate is converted to a heart rate variability (HRV) measure, which is used to designate a current state class using an artificial intelligence (AI) model. The state class is used to automatically retrieve appropriate coaching media content for consumption on the mobile device.

### CROSS-REFERENCE TO RELATED APPLICATIONS

This application claims priority to Provisional U.S. Pat. Application No. 63/268,465, titled “SYSTEM AND METHOD FOR BIOMETRIC AND PSYCHOMETRIC ASSESSMENTS FOR BURNOUT PREVENTION,” filed on Feb. 24, 2022, by Jessica Corbin et al., which is incorporated herein by reference in its entirety and for all purposes.

### TECHNICAL FIELD

The present disclosure relates to computer systems, and specifically to biometric and psychometric analysis using computer systems.

### BACKGROUND

For people in the workforce, burnout is becoming increasingly prevalent. Burnout is a state of emotional, physical, and mental exhaustion caused by excessive and prolonged stress. It occurs when a worker feels overwhelmed, emotionally drained, and unable to meet constant demands. As the stress continues, the worker begins to lose the interest and motivation that led the worker to take on a certain role in the first place. Burnout reduces productivity and saps energy, leaving one feeling increasingly helpless, hopeless, cynical, and resentful. Eventually, a worker may feel like they have nothing more to give.

While employers are not blind to the issue, they know that stress is a problem and they are trying to solve it but they currently lack the insight and the tools to address this problem. With remote work on the rise, not only the stress, but the physical and emotional distance is further exacerbating the problem. Conventionally, workers could only deal with burnout by seeking therapy, or engaging in holistic activities, such as yoga and meditation. However, these approaches rely on personal interactions and subjective psychological assessments. Because of the subjective and personal nature of these conventional remedies, the results may be inconsistent and access may not be readily and widely available. Thus, there is a need for more consistent and objective data driven approach to burnout prevention.

### SUMMARY

The following presents a simplified summary of the disclosure in order to provide a basic understanding of certain embodiments of the present disclosure. This summary is not an extensive overview of the disclosure and it does not identify key/critical elements of the present disclosure or delineate the scope of the present disclosure. Its sole purpose is to present some concepts disclosed herein in a simplified form as a prelude to the more detailed description that is presented later.

Aspects of the present disclosure relate to a system, computer readable medium, and a method for biometric and psychometric based content display. The method includes storing a psychometric assessment instrument as a data structure on a mobile device. The psychometric assessment instrument includes user data for ascertaining a user’s psychometric identity from among a predefined plurality of psychometric classifications. Next, content is collected and tagged by attribute, at a server, for each specific classification of the plurality of psychometric classifications, wherein the content is further organized across three state classes and cross-referenced with a plurality of interests, the state classes and plurality of interests being used to appropriately tag video and audio content with metadata. Then, at a camera on the mobile device, a pulse rate of the user is measured using temporal color contrast between two frames. The pulse rate is then converted into a measure of heart rate variability (HRV) using a predetermined algorithm. Next, a current state class is designated from among the three state classes based on the measure of HRV using an artificial intelligence (AI) model. A structured query is transmitted by the mobile device to the server, based on the psychometric identity, the plurality of interests, and the current state class, in order to receive a machine readable list of matching coaching media content. Last, selected content from the list of matching coaching media content is automatically retrieved from the server for display on the mobile device.

In some embodiments, the plurality of psychometric classifications includes 12 different archetypes. In some embodiments, designating a current state class includes assessing self-reported attributes, the self-reported attributes including: sleep, activity level, nutrition, stress management, and perceived levels of productivity. In some embodiments, the psychometric identity is updated periodically with a re-assessment. In some embodiments, the pulse rate is determined by measuring temporal distance in between peaks. In some embodiments, the three state classes are categorized as Push, Maintain, and Recover. In some embodiments, user data remains on the mobile device and is never sent to the server as raw data.

Additional advantages and novel features of these aspects will be set forth in part in the description that follows, and in part will become more apparent to those skilled in the art upon examination of the following or upon learning by practice of the disclosure.

### BRIEF DESCRIPTION OF THE DRAWINGS

The disclosure may best be understood by reference to the following description taken in conjunction with the accompanying drawings, which illustrate particular embodiments.

FIG. 1 illustrates a system diagram, in accordance with one or more embodiments of the present disclosure.

FIG. 2 illustrates a flow diagram of a process for calculating HRV, in accordance with one or more embodiments of the present disclosure.

FIG. 3 illustrates an example of processing fingerprint images, in accordance with one or more embodiments of the present disclosure.

FIG. 4A illustrates an example of a raw signal captured over time, in accordance with one or more embodiments of the present disclosure.

FIG. 4B illustrates an example of a raw signal after passing through a filter, in accordance with one or more embodiments of the present disclosure.

FIG. 5A illustrates an example of a low frequency drift signal, in accordance with one or more embodiments of the present disclosure.

FIG. 5B illustrates an example of a signal minus the low frequency drift, in accordance with one or more embodiments of the present disclosure.

FIG. 6 illustrates how prominence is defined in a signal, in accordance with one or more embodiments of the present disclosure.

FIG. 7 illustrates an example valley finding algorithm, in accordance with one or more embodiments of the present disclosure.

FIG. 8 illustrates an example of a local valley prominence calculation, in accordance with one or more embodiments of the present disclosure.

FIG. 9 illustrates an example of a false positive in the data, in accordance with one or more embodiments of the present disclosure.

FIG. 10 illustrates a method for biometric and psychometric based content display, in accordance with one or more embodiments of the present disclosure.

FIG. 11 illustrates an example of a computer system, configured in accordance with one or more embodiments of the present disclosure.

### DETAILED DESCRIPTION

Reference will now be made in detail to some specific examples of the disclosure including the best modes contemplated by the inventors for carrying out the disclosure. Examples of these specific embodiments are illustrated in the accompanying drawings. While the disclosure is described in conjunction with these specific embodiments, it will be understood that it is not intended to limit the disclosure to the described embodiments. On the contrary, it is intended to cover alternatives, modifications, and equivalents as may be included within the spirit and scope of the disclosure as defined by the appended claims.

For example, the techniques of the present disclosure will be described in the context of computer systems and network transactions. However, it should be noted that the techniques of the present disclosure apply to a wide variety of network transactions, collaborative environments, data structures, and different types of data. In the following description, numerous specific details are set forth in order to provide a thorough understanding of the present disclosure. Particular example embodiments of the present disclosure may be implemented without some or all of these specific details. In other instances, well known process operations have not been described in detail in order not to unnecessarily obscure the present disclosure.

Various techniques and mechanisms of the present disclosure will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present disclosure unless otherwise noted. Furthermore, the techniques and mechanisms of the present disclosure will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

From a human perspective, one method of burnout prevention involves regenerating human energy in a holistic and data-driven way. The techniques and mechanisms presented herein provide for a data driven and human centered behavior change platform for high performing cultures at risk for burnout.

In some embodiments, in order to measure, track, and treat burnout, systems may utilize a true body mind approach. Thus, biometrics and psychometrics information can be collected, analyzed, and transformed into an effective burnout prevention remedy. According to various embodiments, the data collected is also transformed into effective strategies for changing user behavior. Systems that use only psychometrics suffer from an inability to help people make better choices that support positive outcomes. Using psychometrics alone, most people are not in tuned enough with their physiology to understand what it is they need. Systems that only use biometrics data, without meaning, falls flat. In other words, systems that simply measure data regarding how stressed a user is, but do not communicate how to address this stress, is ultimately ineffective. Thus, a system that overlays both biometric and psychometric data gives a much higher probability of actually addressing the burnout.

According to various embodiments, a platform captures users’ physical and behavioral data to automatically select content for individuals, teams, and organizations. The captured data is then used to provide actionable steps in a way that is accessible and achievable on a daily basis through a mobile device or computer.

In some embodiments, once a user is on boarded onto the platform, the user then takes a one minute assessment that helps the system profile the use into one of twelve types. In some embodiments, this assessment comprises six questions to answer, with each answer involving a scale of 1-7 to answer. In some embodiments, the scores for each question show the user’s highest expression across six dimensions: Honesty-Humility, Emotionally, Extraversion, Agreeableness (versus Anger), Conscientiousness, and Openness to Experience. In some embodiments, this assessment allows the system to categorize the user into an archetype that most likely describes the user. In some embodiments, the classifications include 12 different HEXACO personality archetypes.

In some embodiments, the type is not communicated to the user, but is instead used to facilitate communication with the user via a pre-defined language that resonates with users of that particular type. In some embodiments, using the right language is vital to help motivate the end user. Thus, in some embodiments, different language is used to communicate the same objective depending on the type classification of the user. In some embodiments, profiling also allows the system to transmit pre-determined push notifications to the user, called “nudges,” to motivate in a language that strongly resonates. In some embodiments, the system then utilizes biofeedback driven coaching. In some embodiments, a server stores a vast coaching media library and streaming components, which is accessible via a portal or on the platform. In some embodiments, biofeedback is collected daily from the user and then used to automatically select content for display.

In some embodiments, the system is configured to build a model over time for a user’s psychometric data during a particular cycle. In some embodiments, the model’s framework is a statistical model that assumes a cycle of push, maintain, and recover, which is summarized as a biological processes in humans. In some embodiments, based on the model, the system determines the user’s classification for the day, or over several days, and then categorizes the user’s measurements into the three bins of push, maintain, and recover. In some embodiments, the push category is used for an energetic day. In some embodiments, the maintain category is used for a maintain day. In some embodiments, the recover category is used for self-care.

In some embodiments, the platform can be accessed via a mobile app. While in the app, the user can get a measurement of a biomarker. In some embodiments, heart rate variability (HRV) is one objective biomarker that systems use for assessment of stress. In some embodiments, the user can measure their HRV score. In some embodiments, this measurement can be done by putting a finger to the camera of the mobile device. In some embodiments, the app provides a one minute stress assessment or energy assessment based on the HRV score. Next, depending on the HRV score, the user then receives audio or video content that caters to their energetic needs. The user can get a daily pulse check using this mechanism. According to various embodiments, the HRV scores are measured and stored over time. The system can then monitor the nervous system as a function of the HRV scores over time. In some embodiments, with time and data, the platform becomes more intelligent and predictive, which increases efficacy and efficiency. In some embodiments, after the HRV score is obtained, the system performs a search on the backend, e.g., in a cloud infrastructure, on servers that look at the right file of audio video to play based on the HRV score and cross-referenced with the user’s psychometrics. In some embodiments, this is based on which of 12 archetypes the user falls into, and what the right coaching is in the user’s current situation, e.g., a maintain day. For examples, high extraverts react to a certain set of coaching videos differently than an introvert. Thus, the system is configured to deliver personalization against biometric measurement using psychometric content. Since all stored coaching and other media content is tagged using the archetypes described above, a search based on archetype can be subsequently performed using the determination of where the user is in the push/maintain/recover cycle based on the HRV measurements. In some embodiments, the content is tagged using metadata tags to flag content appropriate for each personality type. In some embodiments, each different archetype has their own library of media that is tagged for certain HRV measurements.

In some embodiments, the system is configured to do a larger assessment or multiple subsequent assessments. In such embodiments, this allows the system to continue to hone the categorization of the users by sending them questions throughout their experience. In some embodiments, the initial assessment being a maximum of one minute in duration is important for efficient onboarding. However, over time, more accuracy may be needed and thus, subsequent assessments allow for deeper understanding of the user. Consequently, in some embodiments, the system prompts the user with questions through their engagement in predetermined intermittent intervals. In some embodiments, the intervals are defined through progress and milestones. In some embodiments, the intervals are based on length of time. In some embodiments, the intervals are based on a combination of milestones and length of time. Thus, in some embodiments, the psychometric portion is done initially and then perhaps intermittently following the initial intake. In some embodiments, the biometrics portion is done daily.

### Description of App

In some embodiments, the application UI includes a welcome screen, a privacy statement upfront, a single sign-on, a psychometric assessment-which can be as simple as six questions with the option to agree/disagree. This information allows the system to understand how the app needs to talk to a particular user. In some embodiments, the system then asks the user what kind of content the user wants to receive. In some embodiments, because of energy management and stress, there may be a sub-categorical emphasis on what the system can slow drift to the user. In some embodiments, the user picks categories. Based on the info, the system assigns the user a coach, but in some embodiments, the user can pick as many coaches as they want. In some embodiments, a coach is not selected, but rather a coaching regimen based on audio and visual content data automatically selected and delivered based on daily biometric data and psychometric assessment data.

In some embodiments, the UI includes a screen where a user can get their energy status. Depending on the user and the biometric information, the energy status can reflect a push day, a maintain day, or a recovery day, along with a raw score. For example, if a day is a push day, the user can then quickly survey their actions for the prior day. For example, the survey can include how did the user sleep or how did the user eat. The survey can include information regarding the perceived level of productivity. Thus, in some embodiments, the user can start making correlations between their choices one day and a different day.

In some embodiments, the app UI includes a screen that shows content play. In such embodiments, this includes health and performance advice based on the user’s HRV score. In some embodiments, the system stores prerecorded media and matches the media with scores according to a predetermined algorithm.

In some embodiments, the content in the media library are tagged based on push, maintain, or recover cycles. In such embodiments, the content is driven by biofeedback. In some embodiments, the user can even search by expertise. The user can go into any of the profiles and see pictures, videos, and biographies. In some embodiments, the system is configured to hold open communication channels between the coaches and the users.

In some embodiments, the app UI includes displays that show a user’s energy trends over time. In some embodiments, the user has the ability to filter by any of multiple criteria, e.g., months, days, duration, etc. For example, the user can filter to see what Tuesday was like and what their subjective answers were on that day. In some embodiments, the system is configured to transform data into trends and predictions over a sufficient period of time.

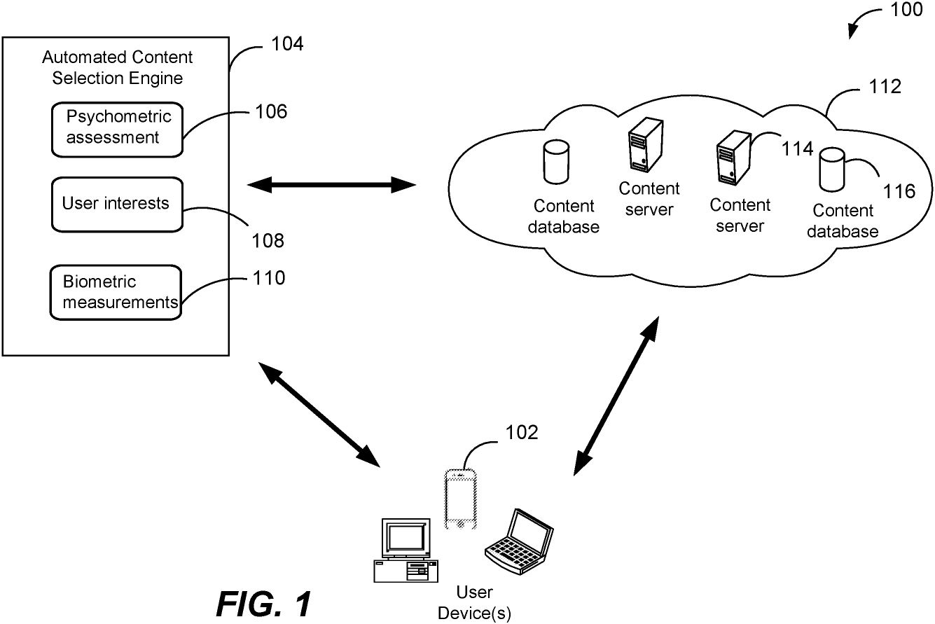

FIG. 1 illustrates a system diagram, in accordance with one or more embodiments of the present disclosure. System 100 illustrates an automated content selection engine 104 that utilizes psychometric assessment 106, user interests 108, and biometric measurements 110 to select specific tagged content from content library 112, which may include content servers 114 and content databases 116, for display on user devices 102, e.g., a mobile device.

In some embodiments, automated content selection engine 104 that drives ideal coaching content to users based on their biometric needs and is optimized through psychometric insight. In some embodiments, through application of this engine and its attendant tools, automated content selection engine 104 identifies the personality of people, coaches, and even the content that a coach provides to their audience. Using this insight, automated content selection engine 104 determines both ideal coaches for people seeking guidance and empowers those coaches to further ensure their content is constructed to effectively communicate to the very people they are training. In some embodiments, automated content selection engine 104 automatically selects content even without selecting a coach.

According to various embodiments, selecting and delivering content based on both deep psychometric understanding and day-to-day biomarkers are core to automated content selection engine 104. In some embodiments, combined physical and behavioral data includes performance coaching enacted by trained professionals and augmented by content selected by automated content selection engine 104 based on biometric measurements, e.g., Heart Rate Variability (HRV). In addition, in some embodiments, automated content selection engine 104 utilizes bespoke assessments and analysis to further hone selection of content. In other words, in some embodiments, automated content selection engine 104 optimizes content selection based on both coach personality and how to best communicate messaging to the audience based on their personalities. In some embodiments, automated content selection engine 104 is configured to use a specific lexicon for different users in order to specifically tailor content creation and messaging that improves efficacy through personality understanding.

According to various embodiments, when introduced to the system platform, users (audience members) are given a six-item personality assessment to gain insight into how best to communicate to them and match them with appropriate coaches. Theses answers are used to form a psychometric assessment 106. In some embodiments, as time progresses, users are given additional questions to support deeper understanding of the facets of their personality to offer stronger insight to the system. These answers are used to form user interests 108.

According to various embodiments, over time, system 100 gains enough information to form a comprehensive view of an individual that is not just limited to the main six dimensions of HEXACO. In some embodiments, system 100 has the detail to break down a user’s personality to sub-dimensions that offer highly-detailed insight into how best to reach them with content.

In some embodiments, in addition to selecting content for user, system 100 also selects a matching coach for the user. In such embodiments, unlike users, coaches must take a 100-item psychometric assessment in order for system 100 to retrieve the dimension and sub-dimensional data immediately in order to optimize the coach’s specific content. In some embodiments, coaches’ and users’ personalities can be used to match coaches with an audience of users with whom they have congruent personalities, thereby helping them make personal connections that can drive increased adoption, loyalty, and trust.

According to various embodiments, automated content selection engine 104 must process the assessed and measured data in order to select the appropriate content form content library 112. In some embodiments, prior to user consumption, each piece of advice content is tagged with a metadata tag and tailored to the user’s personality to maximize the effectiveness of the messaging. In some embodiments, this is done through understanding the coach’s personality and through use of a Psychometric Optimization Lexicon (POL). In some embodiments, the coach’s personality provides the coach with the ability to improve the quality of their content based on tapping into the POL. In some embodiments, the POL is built using the research of psyML to sort out diction, phrases, and communication methodologies that are best suited to both personality archetypes and comprehensive personalities of each individual.

In some embodiments, they system enables and prompts coaches to algorithmically create a dozen or more versions of their content based on key psychometrics archetypes to ensure their message is always tailored based on both their content, their personality, and the personality of the users in their audience. Since content is delivered automatically and directly to users, variations are effectively micro-targeted to individuals.

In some embodiments, system 100 uses an algorithm that applies the POL against coach content such that each piece of content is specifically designed to appeal to the key archetypes for High and Low scores on the six main dimensional scales. In some embodiments, composition of the content variations is handled through automation built into the system. In some embodiments, using the algorithm, scripts for video, audio and written content can be optimized based on psychometrically-tagged diction from the POL and suggested communication methodologies tied to the prominent dimensions of both coach and user. For example, a message sent to a person scored as high in the personality dimension, Openness to Experience, may use the promise of something new to encourage positive behavior. The same message designed for a person whose assessment says they are Low Agreeableness would instead focus on data and proven practices to encourage the same behavior.

According to various embodiments, all data and metadata will be used to implement machine learning models to optimize content selection tools. In some embodiments, in addition to the psychometric details of users and coaches, system 100 may process daily question responses, types of advice, categories of advice, team and individual feedback, the RMP advice that is delivered and how users reacted to it, and the direct user feedback built into the platform to further develop models. In some embodiments, as the models are developed, automated content selection engine 104 evolves to work for a mix of dimensions of HEXACO, in order to avoid simply optimizing on the most dominant traits. In some embodiments, the models are trained to support all six dimensions for each coach and individual so that a mix of diction, phrases, communication techniques, and reactions to various pieces of advice types will work in tandem to deliver ideal messaging and/or content delivery.

In some embodiments, system 100 uses the HEXACO system as the personality inventory used to determine the dimensions for scoring each user. HEXACO allows for a holistic view of the user across the six dimensions that make up the scale. In some embodiments, an initial short-form test provides the algorithm with a basic archetype. In some embodiments, the app also delivers users additional questions over time to get deeper insight into their scoring across the dimensions. Through these finer point questions, system 100 is able to understand the user’s individual scoring based on the four sub-dimensions of the major HEXACO dimensions. In some embodiments, with this deep insight into the user’s personality, the algorithm is able to draw upon a POL of terms and phrases that are used to deliver ideal diction for the coaching content used in the app. A quick overview of HEXACO is provided below.

### HEXACO Dimensions

Honesty-Humility: People with high scores on the Honesty-Humility scale avoid manipulating others for personal gain. Conversely, people with low scores on this scale will flatter others to get what they want and feel a strong sense of self-importance. The sub-dimensions for Honesty-Humility are: Sincerity, Fairness, Greed Avoidance, and Modesty.

Emotionality: People with high scores on the Emotionality scale experience fear of physical dangers and feel empathy and sentimental attachments with others. Those with low scores on this scale are not deterred by the prospect of physical harm and feel emotionally detached from others. The sub-dimensions for Emotionality are: Fearfulness, Anxiety, Dependence, and Sentimentality.

Extraversion: People with high scores on the Extraversion scale feel positively about themselves and experience positive feelings of enthusiasm and energy from other people. People with lower scores on this scale consider themselves unpopular, prefer to be alone, and tend to feel less lively than others do. The sub-dimensions for Extraversion are: Social Self-Esteem, Social Boldness, Sociability, and Liveliness.

Agreeableness: People with high Agreeableness scores forgive wrongs that they suffered and can easily control their temper. People with low scores on this scale hold grudges, are critical of others’ shortcomings, and are stubborn in defending their point of view. The sub-dimensions for Agreeableness are: Forgivingness, Gentleness, Flexibility, and Patience.

Conscientiousness: People with high scores on the Conscientiousness scale are people who organize their time, strive for accuracy and deliberate carefully when making decisions. People with lower scores tend towards a lack of concerned with schedules and make decisions on impulse or with little reflection. The sub-dimensions for Conscientiousness are: Organization, Diligence, Perfectionism, and Prudence.

Openness to Experience: People with high scores in Openness to Experience become absorbed in the beauty of art and nature and take an interest in unusual ideas or people. Conversely, people with low scores on this scale feel little intellectual curiosity, avoid creative pursuits, and dislike unconventional ideas. The sub-dimensions for Openness to Experience are: Aesthetic Appreciation, Inquisitiveness, Creativity, and Unconventionality.

According to various embodiments, automated content selection engine 104 identifies the specific energetic level of users each day by capturing, via the app, biomarker measurements 110 known to effectively track stress and readiness. In some embodiments, the app captures Heart Rate Variability (HRV) through a camera on the user’s mobile device. HRV is a scientifically-validated biomarker used to track the overall resilience of the autonomic nervous system. Low HRV means the user’s body is having a hard time finding equilibrium and managing stress. High HRV is an indication of resilience, wellbeing, health and performance. HRV is used to monitor cardiovascular health, as well as to track and improve athletic performance.

According to various embodiments, to capture HRV each morning, users hold their finger over their camera and the flash for pre-determined duration of time, e.g., about 55 seconds, to get a measure of their HRV through Photoplethysmography (PPG), an optical measurement for heart rate monitoring. In some embodiments, after the PPG is captured, automated content selection engine 104 plugs the data into an algorithm to calculate HRV in a normalized range, e.g., between 40 and 110. In some embodiments, the information is stored locally on the device of the individual user and is not shared to a server. In some embodiments, individual biomarkers are protected in a database separate from the one that houses user Personally-Identifiable Information.

According to various embodiments, the HRV value gives the user a “pulse check” on their energetic needs. In some embodiments, this value is then compared to the baseline performance of the individual user across a pre-determined period of time, e.g., the entire history of usage of system 100 by the user.

In some embodiments, the user may also be given the opportunity to answer a set of questions, e.g., five questions, about their experience during the previous day. In such embodiments, this self-reporting provides additional detail to inform the algorithm, determine the immediate advice, and contribute to long-term baseline calculations. In some embodiments, each question is rated as Great, Good, Okay, and Poor. In some embodiments, the set of questions include, but are not limited to: “How would you rate your sleep last night?”, “How would you rate your food choices yesterday?”, “How would you rate your activity level yesterday?”, “How well did you manage stress yesterday?”, “How would you rate your productivity yesterday?”.

According to various embodiments, based on that energetic level, the algorithm suggests the appropriate Advice type for the day. In some embodiments, daily coaching advice empowers users to leverage their personal readiness based on the interests and personality of the user, with a focus on actionable insight designed for immediate use. In some embodiments, this scoring system is called “RMP,” which is an abbreviation of the three options it includes: Recover, Maintain, and Push.

In some embodiments, “Recover” is reported if the user is generally below baseline or falls into categories determined to suggest low energy. As a result, the user is given advice in the form of stored content generated from one of their designated coaches that specializes in categories they have selected. In some embodiments, this advice includes text, audio, and/or video content automatically delivered to suggest ways for the user to implement self-care, rest, and other means of not straining their system so they can get back on the road to reclaiming their energy.

In some embodiments, “Maintain” is reported if the user is in sync with their typical baseline for HRV or at a relative energy level that means they are on-track for a normal day. The content provided by the system is then directed toward maintenance of a healthy lifestyle and small changes that can still have a transformative effect on the way users leverage the value of their energy level for the day.

In some embodiments, “Push” is reported if the automated content selection engine 104 detects a value that exceeded the user’s baseline by a significant margin or that tops out the algorithm against typical numbers for the user. The content selected directs users to find ways to leverage the additional energy, whether it is to lean into a work schedule, add more strenuous exercise, or otherwise push the limits of what was planned for the day in order to take advantage of a day when the user is particularly well-resourced and the chance of burnout is smaller.

According to various embodiments, system 100 uses the RMP cycle in order to increase performance over time, e.g., increase HRV baselines over time, through use of the app and automatically selected content on a daily basis. In some embodiments, HRV is variable based on individuals so a raw score, devoid of historical context, is insufficient for determining an RMP stage. Thus, in such embodiments, the algorithm takes into account a variety of factors for calculating useful biomarkers.

According to various embodiments, automated content selection engine 104 calculates the RMP stage using an algorithm. One example algorithm may be the following:

$$\overline{H R {V}_{i}} = \frac{1}{7} {\sum }_{t = 7}^{7} H R {V}_{i t}$$

$$R M {P}_{i t} = \left|\begin{matrix}\text{recover} & \text{if} H R {V}_{t i} < \overline{H R {V}_{i}} - 0.5 ∗ \sqrt{\frac{\sum {\left(H R {V}_{i t} - \overline{H R {V}_{i}}\right)}^{2}}{6}} \\ \text{push} & \text{if} H R {V}_{t i} < \overline{H R {V}_{i}} + \sqrt{\frac{\sum {\left(H R {V}_{i t} - \overline{H R {V}_{i}}\right)}^{2}}{6}} \\ \text{maintain} & \text{else}\end{matrix}\right)$$

In the above algorithm, the RMP value is “recover” if the HRV is less than the mean HRV over the last 7 days minus one HRV Standard Deviation. The RMP value is “push” if the HRV is greater than the mean HRV over the last seven days plus half of a HRV Standard Deviation. The RMP value is “maintain” for all other HRV values.

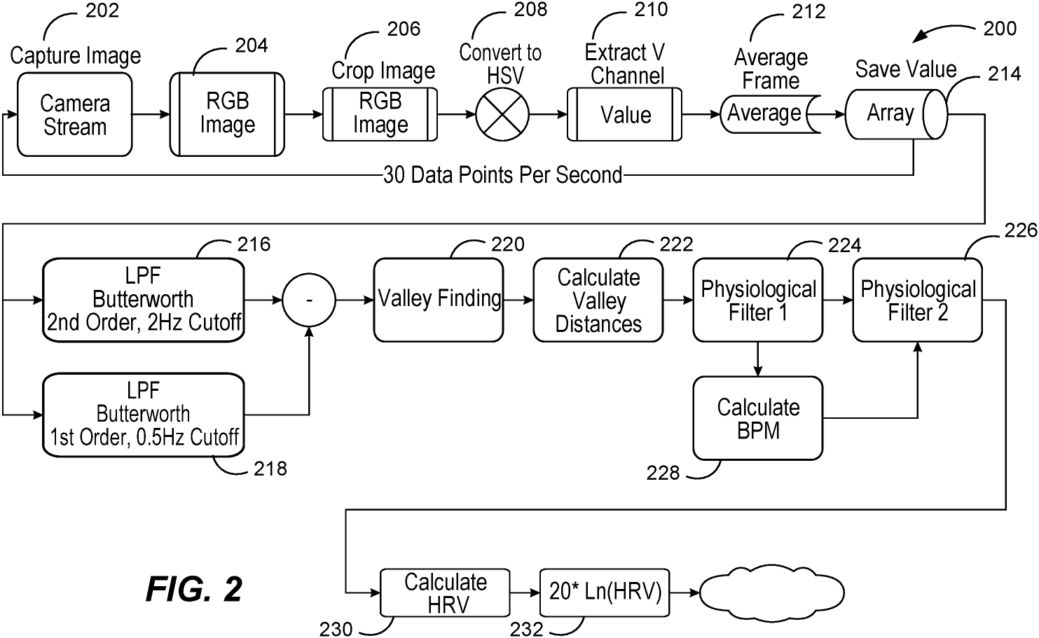

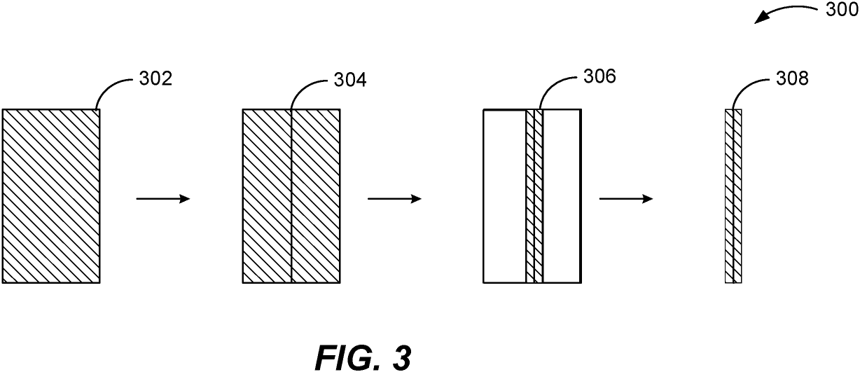

FIG. 2 illustrates a flow diagram of a process for calculating HRV, in accordance with one or more embodiments of the present disclosure. Process 200 begins with capturing (202) an image from the camera of the mobile device. In some embodiments, capturing the image includes capturing a stream of images, or a series of image frames, where each frame is saved separately to memory. In some embodiments, each image is an RGB (Red Green Blue) image 204. In some embodiments, because cameras have such high definition in today’s technology, in order to speed up processing, each RGB image needs to be cropped into a cropped RG image 206. As discussed below with reference to FIG. 3, in some embodiments, cropped image 206 need only be 10 pixels wide in order to function properly for the algorithm. At step 208, cropped image 206 is then converted into HSV (Hue Saturation Value), which is a color space that allows the algorithm to get extract more data out of the cropped image. This is because RGB does not give enough data. In some embodiments, because the heart rate (HR) is most clearly illustrated in the V channel of HSV, the V channel is extracted at step 210. In some embodiments, the pixel values of each frame are then averaged (212) into a single data point. In some embodiments, each average frame value forms a singular point on a plot graph to determine heart rate. At step 214, the average frame value is stored in an array. In some embodiments, steps 202 through 214 are repeated until the data array includes enough data point values. In some embodiments, the algorithm extracts 30 data points per second over a span of about 55 seconds.

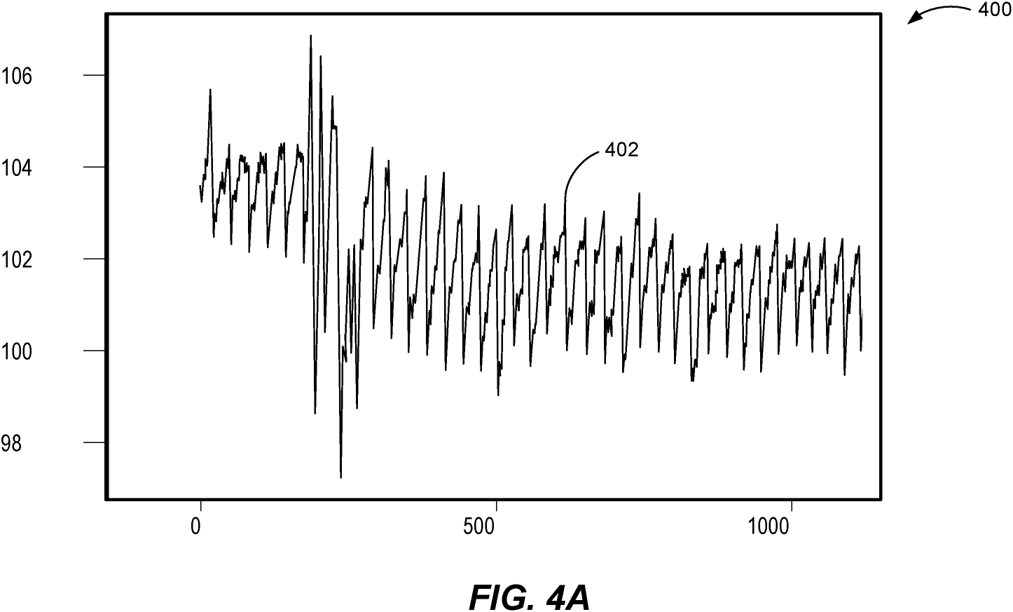

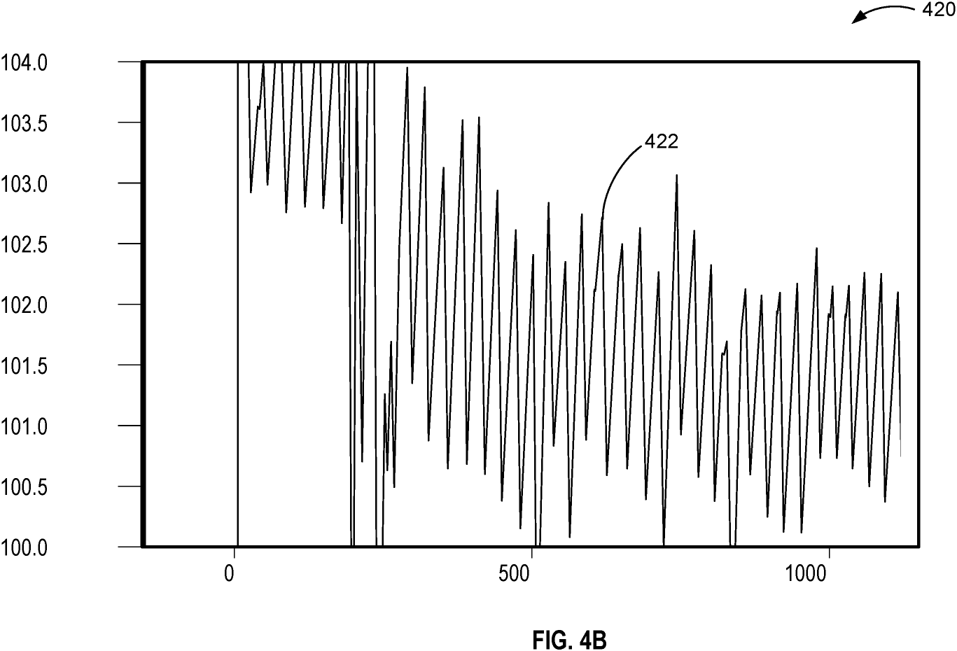

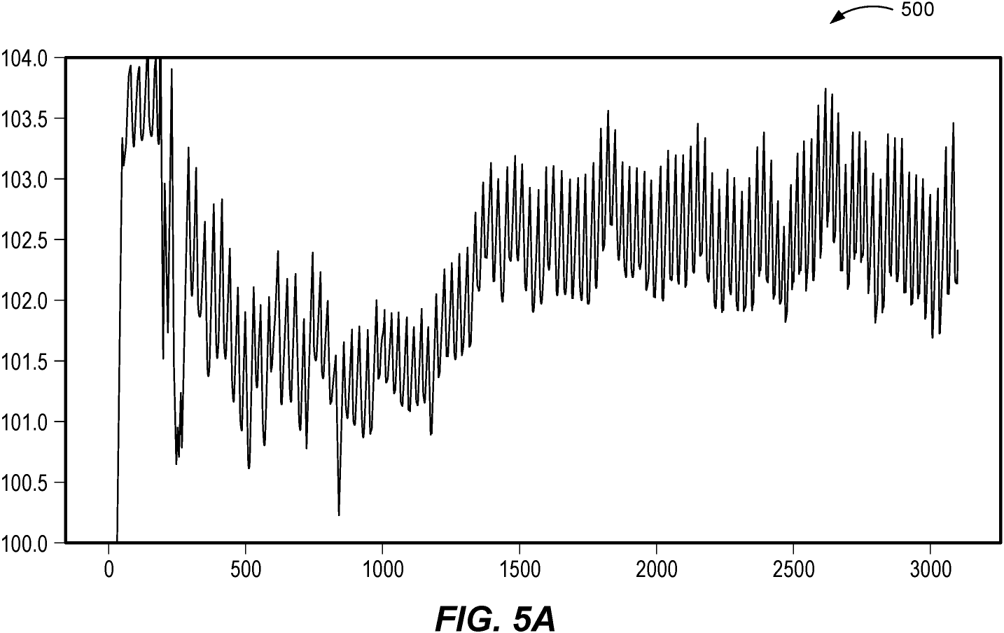

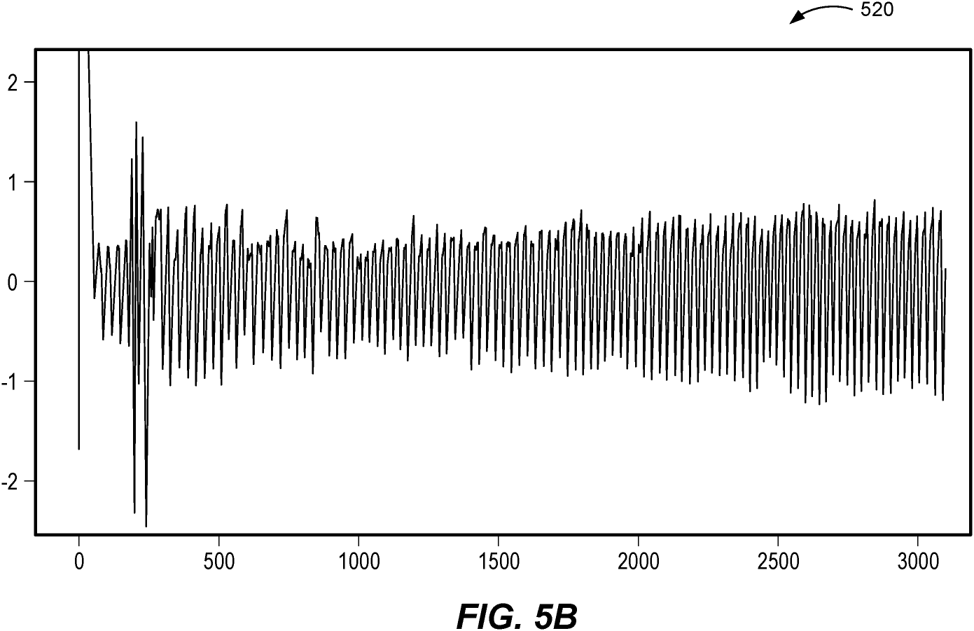

In some embodiments, once the array has been filled, the total number of points, referred to as a “signal,” is then passed through one or more filters to account for drift in the data. As shown in FIG. 2, in some embodiments, the signal is passed through two filters, a low pass filter (LPF) 216 and a second LPF 218, that essentially work together as a band pass filter. In some embodiments, LPF 218 is a first order low pass filter with a 0.5 Hz cutoff to remove frequencies that are too low. An example of the output of LPF 218 is illustrated in FIGS. 5A-5B. In some embodiments, LPF 216 is second order low pass filter with a 2 Hz cut off to remove frequencies that are too high. and there’s height and noise. An example of the output of LPF 218 is illustrated in FIGS. 4A-4B. In some embodiments, the algorithm is only interested in signals that correspond to a HR in the range of 50-300 BPM, anything outside of that gets removed by the low pass filters. In some embodiments, the resulting filtered signal is the difference of the output of LPF 216 minus the output of LPF 218.

In some embodiments, after the signal has been filtered, the algorithm then finds (220), or identifies, some of the valleys in the signal. In some embodiments, the algorithm then calculates (222) the distances between adjacent valleys in order to determine a heart rate. In some embodiments, the determined HR may pass through one or more physiological filters to remove any data points that do not correlate to a HR that is not physiologically possible. As shown in FIG. 2, process 200 includes two physiological filters, physiological filter 224 and physiological filter 226. In some embodiments, physiological filter 224 operates by to normalize HR BPM to human ranges. In some embodiments, physiological filter 226 acts to further refine the HR BPM. In some embodiments, the physiological filters calculate (228) the BPM by calculating the distance between peaks in the signal, which may translate to a time duration in ms.

In some embodiments, process 200 then proceeds to calculate (230) the HRV from the HR BPM. At step 232, the calculated HRV value is then normalized by taking the Ln of the HRV and multiplying the result by 20 (20*Ln(HRV)). In some embodiments, step 232 puts the HRV values onto a linear scale and brings the HRV value to under 100, in order to use numbers that appear more like a percentage for the purposes of making the HRV more understandable to the user.

FIG. 3 illustrates an example of processing fingerprint images, in accordance with one or more embodiments of the present disclosure. Process 300 illustrates just one example method for the cropping image step 206 in FIG. 2. Process 300 begins with the user putting a finger over the camera and flash. An image 302 is captured by the camera. In some embodiments, image 302 is an image of the user’s finger as illuminated by the flash. In some embodiments, the idea is that as blood pulses through the finger, the illumination of the pixels changes because blood causes the blood to expand. The expansion of the blood vessels in the finger cause less light to be captured in the image. Thus, during a pulse (which can be used as a proxy for a heart beat), the overall value of the pixels is “darker,” or of lower value. Consequently, valleys can be used to represent heat beats.

In some embodiments, image 302 represents a full frame captured by the camera. Next, the algorithm finds the center line 304 of the image. Then, the algorithm takes a small pre-determined number of pixels 306 to the left and to the right of center line 304. In some embodiments, the width of this rectangular subset of pixels is around 10 pixels wide. Last, the rectangular subset is cropped out to form cropped image 308. In some embodiments, cropped image 308 is an example of cropped image 206 in FIG. 2.

FIG. 4A illustrates an example of a raw signal captured over time, in accordance with one or more embodiments of the present disclosure. As mentioned above, LPF filters are used to remove noise and smooth the signal for peak (or valley) extraction, which is used to estimate heartbeats. FIG. 4A shows a raw signal 400 before passing through a LPF filter. As shown in FIG. 4A, peaks 402 of raw signal 400 look “jagged,” with a lot of noise.

FIG. 4B illustrates an example of a raw signal after passing through a filter, in accordance with one or more embodiments of the present disclosure. FIG. 4B shows the result of passing raw signal 400 through a second order LPF filter with a 2 Hz cutoff, such as LPF filter 216. As shown in FIG. 4B, filtered signal 420 has peaks 422 that look much smoother, or with less noise, than peaks 402.

FIG. 5A illustrates an example of a low frequency drift signal, in accordance with one or more embodiments of the present disclosure. As mentioned above, a second low pass filter is run on the signal to remove low frequencies. The resulting filtered signal from the second low pass filter is then subtracted from the filtered signal from the first low pass filter that removed high frequencies. The difference between the two filtered signals is then used for further processing. FIG. 5A shows raw signal 400 being passed through a first order LPF filter with a 0.5 Hz cutoff, such as LPF filter 218, resulting in filtered signal 500.

FIG. 5B illustrates an example of a signal minus the low frequency drift, in accordance with one or more embodiments of the present disclosure. Difference signal 520 is the result of subtracting filtered signal 500 from filtered signal 420. As shown in FIG. 5B, difference signal 520 does not have the “wavy” nature of filtered signal 500, but instead looks relatively “straight.” This allows for easier processing downstream. As mentioned above, the next step is to find valleys in order to determine the heart rate.

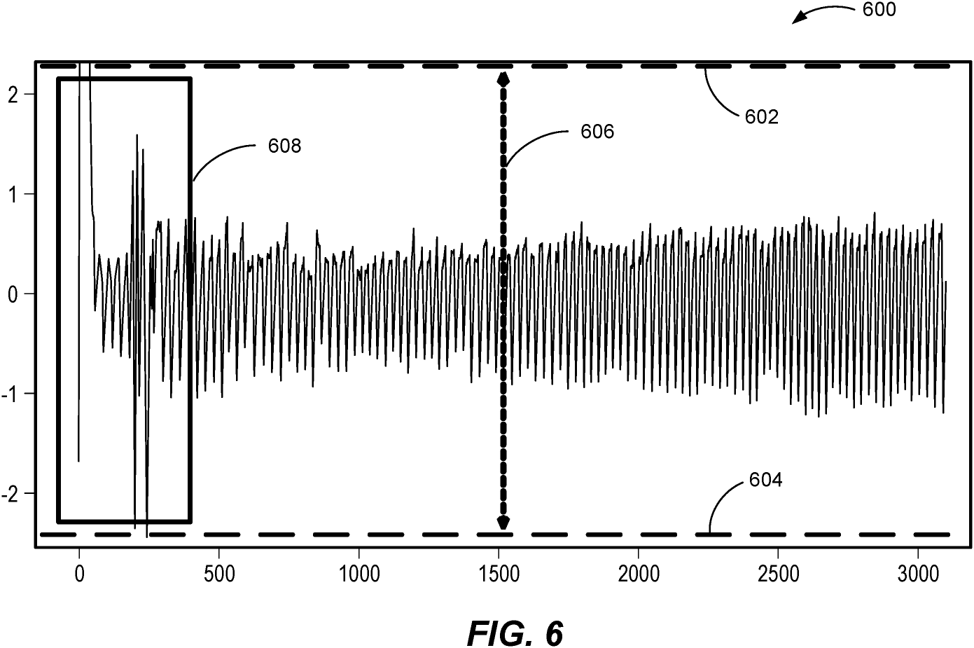

FIG. 6 illustrates how prominence is defined in a signal, in accordance with one or more embodiments of the present disclosure. As shown in FIG. 6, a signal 600 is used to find valleys using a process call “prominence calculation.” In a prominence calculation, each major peak is a heartbeat. Because signal 600 includes many little peaks in between major peaks, the algorithm needs to differentiate between an actual heartbeat and noise. In some embodiments, this is addressed by setting a minimum height threshold for a peak in order for the peak to count as a heartbeat. In some embodiments, this may be accomplished by taking the whole data set, e.g., the entire signal 600, and finding the highest (602) and lowest (604) points and say that the height of a candidate peak needs to be higher than a predetermined percentage, e.g., 10%, of the difference 606 between the highest point 602 and the lowest point 604, in order to be considered a valid peak. This predetermined percentage is the minimum height threshold for evaluating peaks.

In some embodiments, while this approach works well, there can be instances where an outlier portion of signal 600 includes many peaks much higher than the rest of the data, as shown in portion 608 of signal 600. In such embodiments, this outlier portion may lead to a reduction of the algorithm’s sensitivity. Thus, in some embodiments, special filters or normalizing techniques can be applied to outlier sections of signal 600 in order to enhance algorithmic sensitivity and to reduce the frequency of false positive valley detections.

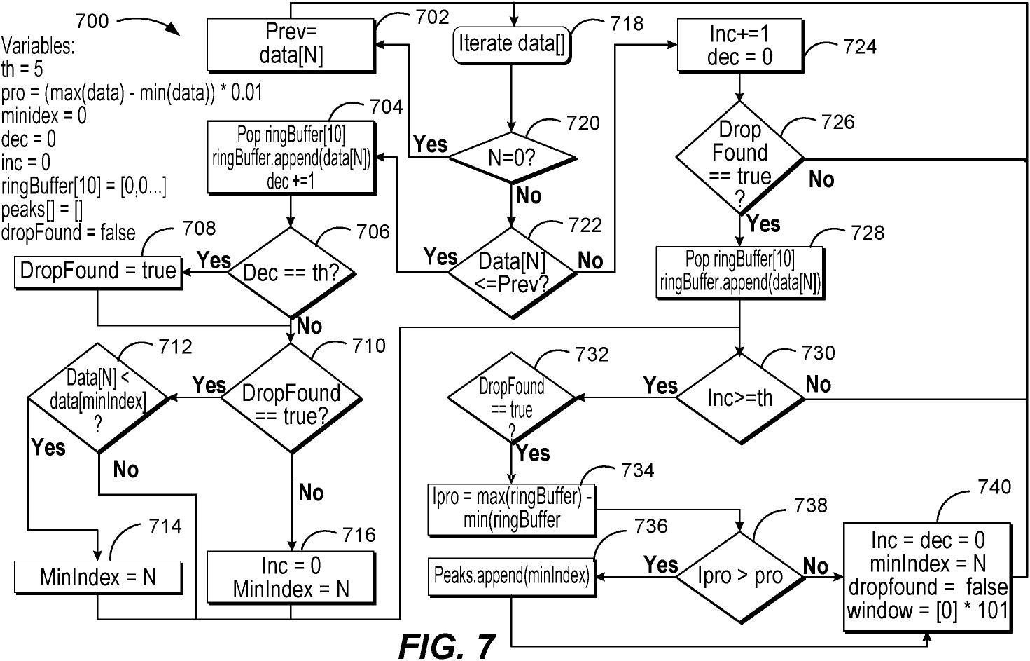

In some embodiments, once signal 600 has been prepared, the automated content selection engine engages a valley finding algorithm, as illustrated in FIG. 7. FIG. 7 illustrates an example valley finding algorithm, in accordance with one or more embodiments of the present disclosure. Using the data array that stores all point values int the signal, valley finding algorithm 700 iterates (718) through each data point. During iteration, algorithm 700 first checks to see the current data point (N) is the first data point in the array (720). If so, then the previous data point is set to equal this data point (702). This is because the algorithm always compares (722) the current data point to the last data point to see if data is increasing or decreasing in value. If the previous data point is higher than N, then the algorithm knows the data is descending. In some embodiments, the algorithm is configured to look for 5 points going down in a row and 5 points going up in a row, resulting in 10 data points in total. Thus, in some embodiments, a ring buffer that can hold ten data points is utilized for this purpose.

Referring back to algorithm 700, if N (current data point) is < or = the previous data point, then the oldest value of the ring buffer is popped (removed), the current data point N is appended to the ring buffer (added), and a “decrease” counter is increased. Again, in some embodiments, the algorithm wants to count how many times the data points have gone down in direct succession and how many time the data has gone up. Next, at 706, the decrease counter is checked to see if the threshold (th) has been met. In some embodiments, since the goal is to find 5 descending/increasing points in a row, the threshold can be set to 5. If decrease counter reaches the threshold of 5, a “dropFound” value is set (708) to “true.” This signals that 5 decreasing points in a row have been found. If the decrease counter has not met the threshold, then the algorithm first checks (710) if the dropFound is already set to true.

If at step 710, the dropFound is already set to true, then the algorithm checks (712) if the data point N is less than the current minimum Index value (minIndex). If so, then N is set as the new minIndex (714) and the algorithm proceeds to the next step, which is step 730. If not, then the algorithm just proceeds to the next step. If at step 710, the dropFound is not already set to true, then the increase counter is set to “0” (because in this part of the loop, N is already less than the previous value), and the minIndex is set to N at step 716. Once 5 descending points have been discovered, the algorithm will then look for 5 ascending points in a row.

At step 730, the increase counter is checked to see if it is greater than or equal to the threshold “th.” If the increase counter is not greater or equal to the threshold, then the algorithm moves on to the next point. If the increase counter is greater or equal to the threshold, then dropFound is checked (732) to see if it has already been set to true. If dropFound is already set to true, then local prominence is set (734) to be the difference in height from the max of the ringBuffer and the min of the ringBuffer. In other words, a possible candidate peak has been identified. Next, at 738, local prominence (candidate peak) is compared to the prominence value set in the beginning of the process (the minimum peak threshold heigh). If the local prominence is greater than the threshold prominence value, then the a true peak has been found and the new peak is added (736) to the list. If the local prominence height is less than the threshold height, then it is a false peak and not included in the peaks list. Thus, in the case that peak is a false peak, everything is reset (740) and the algorithm starts over.

Going back to step 722, if the current data point N is not less than or equal to the previous point, then that means the current point is ascending from the previous point. If that is the case, then in step 724, the increase counter is incremented by 1 and the decrease counter is set to 0. If the dropFound is set to true, then, at step 728, the last oldest value of the ring buffer is popped and the current data N is added to the ring buffer. This means that we found a point that can be counted as one of the 5 ascending points because we already have 5 descending points in the buffer. If the dropFound is not set to true, then that means we have an ascending point without finding a 5 descending points, which the algorithm ignores and moves on to the next point. This process ensures that only ascending points that following 5 descending points are added to the buffer. In other words, the algorithm only populates the ring buffer with ascending points if dropFound is true. By contrast, the algorithm always saves values going down. The difference in treatment is to improve efficiency of the algorithm so that the system does not react to each point unless they are meaningful.

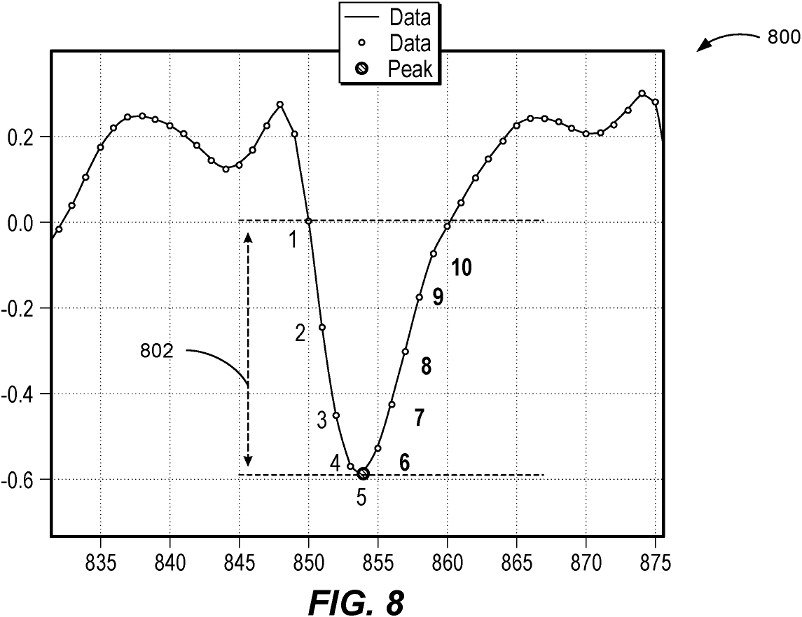

FIG. 8 illustrates an example of a local valley prominence calculation, in accordance with one or more embodiments of the present disclosure. Graph 800 illustrates a visual example of the valley finding algorithm described above with reference to FIG. 7. In FIG. 8, a local prominence 802 is shown to have a height equal to the difference between the highest point in the 10 point ring buffer and the lowest point in the ring buffer.

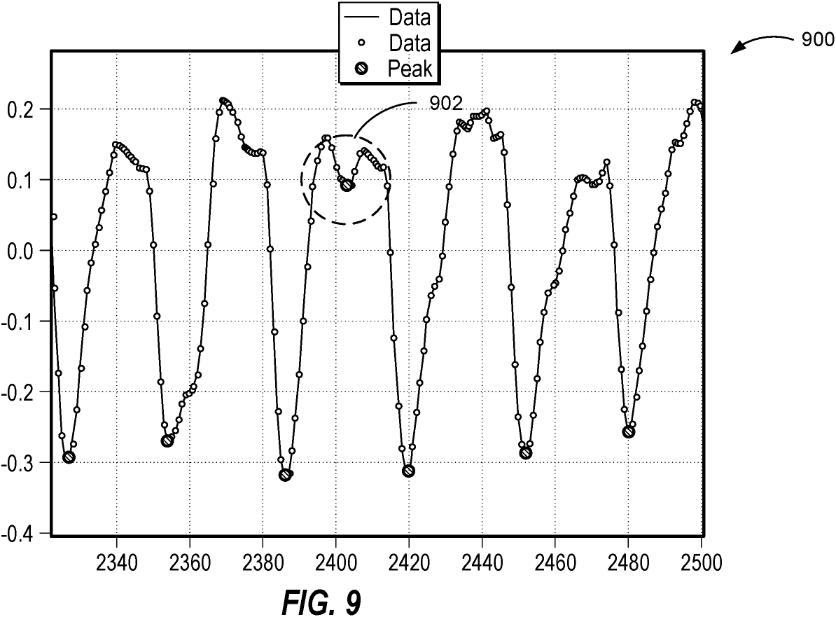

As mentioned above, they system passes signals through one or more physiological filters in order to remove peaks that do not indicate a human heartbeat. FIG. 9 illustrates an example of a false positive in the data, in accordance with one or more embodiments of the present disclosure. Graph 900 shows a peak 902 that passes the prominence minimum threshold used in the valley finding algorithm described above, but yet does not represent a human heartbeat. Thus, the physiological filters work to remove these aberrations from the actual heartbeat signal.

Although many of the components and processes are described above in the singular for convenience, it will be appreciated by one of skill in the art that multiple components and repeated processes can also be used to practice the techniques of the present disclosure.

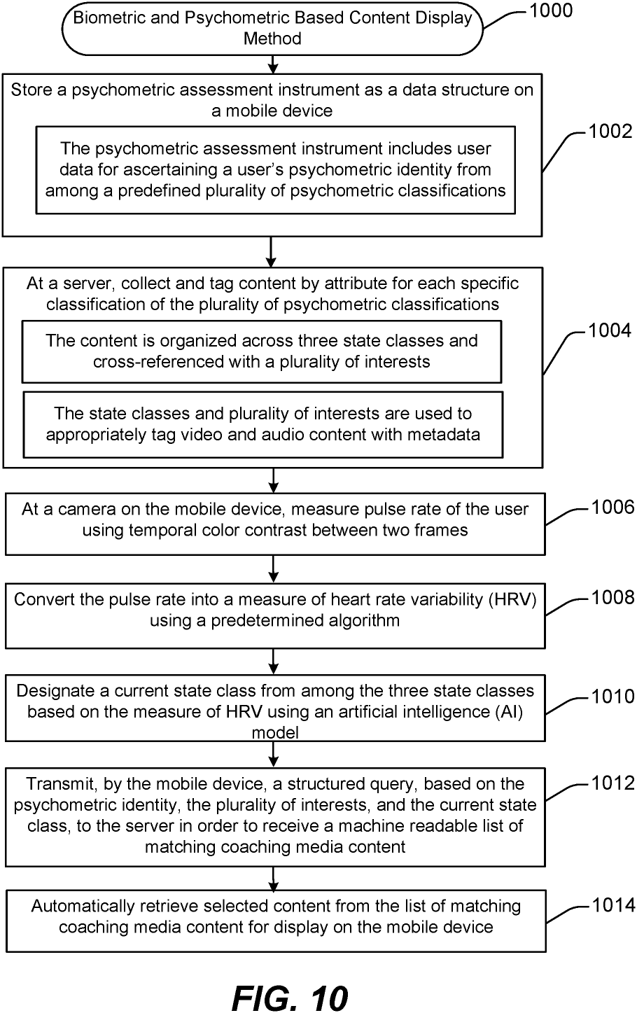

FIG. 10 illustrates a method for biometric and psychometric based content display, in accordance with one or more embodiments of the present disclosure. Method 1000 begins with storing (1002) a psychometric assessment instrument as a data structure on a mobile device. In some embodiments, the psychometric assessment instrument includes user data for ascertaining a user’s psychometric identity from among a predefined plurality of psychometric classifications.

At step 1004, method 1000 includes collecting and tagging content, at a server, by attribute for each specific classification of the plurality of psychometric classifications. In some embodiments, the content is further organized across three state classes and cross-referenced with a plurality of interests. In some embodiments, the state classes and plurality of interests are used to appropriately tag video and audio content with metadata.

At step 1006, method 1000 includes measuring, at a camera on the mobile device, pulse rate of the user using temporal color contrast between two frames. Next, at step 1008, method 1000 includes converting the pulse rate into a measure of heart rate variability (HRV) using a predetermined algorithm. Then, at step 1010, method 1000 includes designating a current state class from among the three state classes based on the measure of HRV using an artificial intelligence (AI) model. Next, at step 1012, method 1000 includes transmitting, by the mobile device, a structured query, based on the psychometric identity, the plurality of interests, and the current state class, to the server in order to receive a machine readable list of matching coaching media content. Last, at step 1014, method 1000 includes automatically retrieving selected content from the list of matching coaching media content for display on the mobile device.

In some embodiments, the plurality of psychometric classifications includes 12 different archetypes. In some embodiments, designating a current state class includes assessing self-reported attributes, the self-reported attributes including: sleep, activity level, nutrition, stress management, and perceived levels of productivity. In some embodiments, the psychometric identity is updated periodically with a re-assessment. In some embodiments, the pulse rate is determined by measuring temporal distance in between peaks. In some embodiments, the three state classes are categorized as Push, Maintain, and Recover. In some embodiments, user data remains on the mobile device and is never sent to the server as raw data.

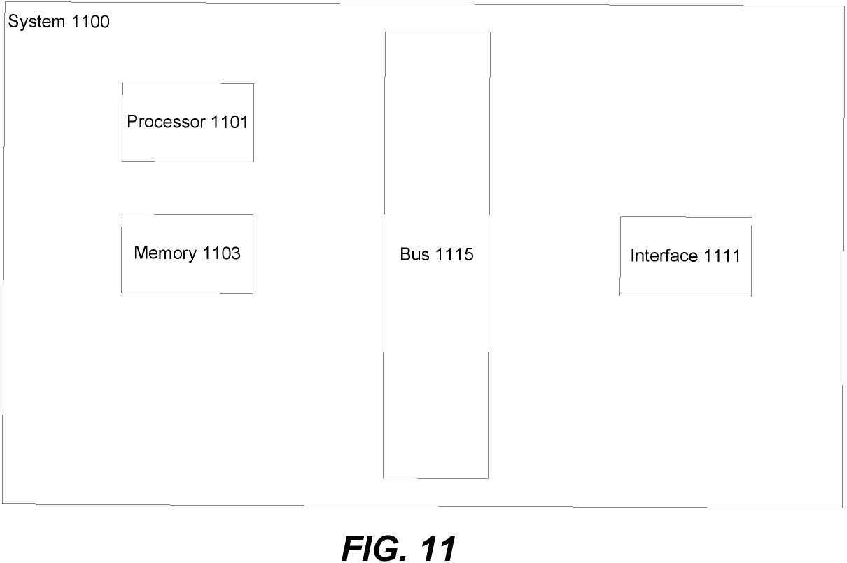

The examples described above present various features that utilize a computer system, such as a mobile device. However, embodiments of the present disclosure can include all of, or various combinations of, each of the features described above. FIG. 11 illustrates one example of a computer system, in accordance with embodiments of the present disclosure. According to particular embodiments, a system 1100 suitable for implementing particular embodiments of the present disclosure includes a processor 1101, a memory 1103, an interface 1111, and a bus 1115 (e.g., a PCI bus or other interconnection fabric). When acting under the control of appropriate software or firmware, the processor 1101 is responsible for implementing applications such as an operating system kernel, a containerized storage driver, and one or more applications. Various specially configured devices can also be used in place of a processor 1101 or in addition to processor 1101. The interface 1111 is typically configured to send and receive data packets or data segments over a network.

Particular examples of interfaces supported include Ethernet interfaces, frame relay interfaces, cable interfaces, DSL interfaces, token ring interfaces, and the like. In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control communications-intensive tasks such as packet switching, media control and management.

According to various embodiments, the system 1100 is a computer system configured to run an automated biometric and psychometric based content retrieval system, as shown herein. In some implementations, one or more of the computer components may be virtualized. For example, a physical server may be configured in a localized or cloud environment. The physical server may implement one or more virtual server environments in which the content retrieval system is executed. Although a particular computer system is described, it should be recognized that a variety of alternative configurations are possible. For example, the modules may be implemented on another device connected to the computer system.

In the foregoing specification, the present disclosure has been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of present disclosure.

What is claimed is:

1. A method comprising:
storing a psychometric assessment instrument as a data structure on a mobile device, the psychometric assessment instrument including user data for ascertaining a user’s psychometric identity from among a predefined plurality of psychometric classifications;
at a server, collecting and tagging content by attribute for each specific classification of the plurality of psychometric classifications, wherein the content is further organized across three state classes and cross-referenced with a plurality of interests, the state classes and plurality of interests being used to appropriately tag video and audio content with metadata;
at a camera on the mobile device, measuring pulse rate of the user using temporal color contrast between two frames;
converting the pulse rate into a measure of heart rate variability (HRV) using a predetermined algorithm;
designating a current state class from among the three state classes based on the measure of HRV using an artificial intelligence (AI) model;
transmitting, by the mobile device, a structured query, based on the psychometric identity, the plurality of interests, and the current state class, to the server in order to receive a machine readable list of matching coaching media content; and
automatically retrieving selected content from the list of matching coaching media content for display on the mobile device.

2. The method of claim 1, wherein the plurality of psychometric classifications includes 12 different archetypes.

3. The method of claim 1, wherein designating a current state class includes assessing self-reported attributes, the self-reported attributes including: sleep, activity level, nutrition, stress management, and perceived levels of productivity.

4. The method of claim 1, wherein the psychometric identity is updated periodically with a re-assessment.

5. The method of claim 1, wherein the pulse rate is determined by measuring temporal distance in between peaks.

6. The method of claim 1, wherein the three state classes are categorized as Push, Maintain, and Recover.

7. The method of claim 1, wherein user data remains on the mobile device and is never sent to the server as raw data.

8. A system comprising:
a server, the server configured for:
collecting and tagging content by attribute for each specific classification of a predefined plurality of psychometric classifications, wherein the content is further organized across three state classes and cross-referenced with a plurality of interests, the state classes and plurality of interests being used to appropriately tag video and audio content with metadata; and
a mobile device, the mobile device configured for:
storing a psychometric assessment instrument as a data structure, the psychometric assessment instrument including user data for ascertaining a user’s psychometric identity from among the predefined plurality of psychometric classifications;
measuring pulse rate of the user, at a camera on the mobile device, using temporal color contrast between two frames;
converting the pulse rate into a measure of heart rate variability (HRV) using a predetermined algorithm;
designating a current state class from among the three state classes based on the measure of HRV using an artificial intelligence (AI) model;
transmitting, by the mobile device, a structured query, based on the psychometric identity, the plurality of interests, and the current state class, to the server in order to receive a machine readable list of matching coaching media content; and
automatically retrieving selected content from the list of matching coaching media content for display on the mobile device.

9. The system of claim 8, wherein the plurality of psychometric classifications includes 12 different archetypes.

10. The system of claim 8, wherein designating a current state class includes assessing self-reported attributes, the self-reported attributes including: sleep, activity level, nutrition, stress management, and perceived levels of productivity.

11. The system of claim 8, wherein the psychometric identity is updated periodically with a re-assessment.

12. The system of claim 8, wherein the pulse rate is determined by measuring temporal distance in between peaks.

13. The system of claim 8, wherein the three state classes are categorized as Push, Maintain, and Recover.

14. The system of claim 8, wherein user data remains on the mobile device and is never sent to the server as raw data.

15. A non-transitory computer readable medium storing instructions to cause a processor to execute a method, the method comprising:
storing a psychometric assessment instrument as a data structure on a mobile device, the psychometric assessment instrument including user data for ascertaining a user’s psychometric identity from among a predefined plurality of psychometric classifications;
at a server, collecting and tagging content by attribute for each specific classification of the plurality of psychometric classifications, wherein the content is further organized across three state classes and cross-referenced with a plurality of interests, the state classes and plurality of interests being used to appropriately tag video and audio content with metadata;
at a camera on the mobile device, measuring pulse rate of the user using temporal color contrast between two frames;
converting the pulse rate into a measure of heart rate variability (HRV) using a predetermined algorithm;
designating a current state class from among the three state classes based on the measure of HRV using an artificial intelligence (AI) model;
transmitting, by the mobile device, a structured query, based on the psychometric identity, the plurality of interests, and the current state class, to the server in order to receive a machine readable list of matching coaching media content; and
automatically retrieving selected content from the list of matching coaching media content for display on the mobile device.

16. The non-transitory computer readable medium of claim 15, wherein the plurality of psychometric classifications includes 12 different archetypes.

17. The non-transitory computer readable medium of claim 15, wherein designating a current state class includes assessing self-reported attributes, the self-reported attributes including: sleep, activity level, nutrition, stress management, and perceived levels of productivity.

18. The non-transitory computer readable medium of claim 15, wherein the psychometric identity is updated periodically with a re-assessment.

19. The non-transitory computer readable medium of claim 15, wherein the pulse rate is determined by measuring temporal distance in between peaks.

20. The non-transitory computer readable medium of claim 15, wherein the three state classes are categorized as Push, Maintain, and Recover.
